(19) United States
(12) Patent Application Publication
(10) Pub. No.: US 2008/0260665 A1
(43) Pub. Date: Oct. 23, 2008

(54) Nail varnish comprising at least one polycondensate and at least one polyoxyalkylene copolymer

(76) Inventors: Laurence Guerchet, Chevilly-Larue (FR); Lizabeth-Anne Coffey-Dawe, Aulnay Sous Bois (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/007,200

(22) Filed: Jan. 8, 2008

Related U.S. Application Data
(60) Provisional application No. 60/879,025, filed on Jan. 8, 2007.

(51) Int. Cl. A61K 8/90; A61Q 3/02

(52) U.S. Cl. 424/61

(57) ABSTRACT

The subject-matter of the present invention is a nail varnish composition comprising, in an organic solvent medium:
a) at least one polycondensate capable of being obtained by reaction:
   of at least one polyol comprising 3 to 6 hydroxyl groups;
   of at least one saturated or unsaturated, linear, branched and/or cyclic, nonaromatic monocarboxylic acid comprising 6 to 32 carbon atoms;
   of at least one aromatic monocarboxylic acid comprising 7 to 11 carbon atoms;
   of at least one saturated or unsaturated, indeed even aromatic, linear, branched and/or cyclic, polycarboxylic acid comprising at least 2 COOH carboxyl groups and/or a cyclic anhydride of such a polycarboxylic acid, and
b) at least one polyoxyalkylene block copolymer chosen from:
   block copolymers of following formula (I):

HO—(CH₂—CH₂—O)ₙ—(CₓH₂ₓO)ₘ—(CH₂—CH₂—O)ₚ—H,
in which n, m and p are independently integers ranging from 1 to 1000 and x is an integer strictly greater than 2 and less than or equal to 10,
copolymers comprising at least one polyethylene glycol block and at least one polypropylene glycol block, the said copolymer exhibiting a molecular weight of greater than or equal to 1000 g/mol and their mixtures.

The subject-matter of the present invention is a nail varnish comprising a polyoxyalkylene block copolymer and a polycondensate of modified alkyd type. Another subject-matter of the invention is a process for making up or caring for the nails.

The nail varnish composition can be employed as varnish base or base coat, as product for making up the nails, as finishing composition, also known as top coat, to be applied to the product for making up the nails, or also as product for the cosmetic care of the nails. These compositions can be applied to human nails or also to false nails.

Nail varnish compositions generally comprise solid particles, such as pigments, pearlescent agents or fillers, which are in dispersion in the aqueous continuous medium or the organic solvent medium of the composition.

In point of fact, these particles have a tendency to settle out over time due to their density, which is greater than that of the continuous medium in which they are dispersed. This settling out is referred to by a modification in the macroscopic appearance of the composition, and in particular, in the case of coloured nail varnishes, by a lack of homogeneity in the colour of the varnish and a deterioration in the hold of the varnish film on the nails.

The formulator has available thickening or gelling agents, such as, for example, bentone, in order to improve the stabilization of the compositions; however, the incorporation of these agents in an amount necessary for the stabilization of the varnish has a tendency to render the varnish film matt, which is not desirable in the case of nail varnishes, where it is desired to obtain a glossy film on the nail.

The Applicant has discovered, surprisingly, that the combination, in a nail varnish possessing an organic solvent medium, of a specific polycondensate of modified alkyd type and of a specific polyoxyalkylene block copolymer makes it possible to obtain a nail varnish composition which exhibits good stability and good homogeneity of the colour over time and also to form a film on the nails which is very glossy and which exhibits good hold over time.

More specifically, a subject-matter of the invention is a nail varnish composition comprising, in an organic solvent medium:
a) at least one polycondensate capable of being obtained by reaction:
   of 15 to 30% by weight, with respect to the total weight of the polycondensate, of at least one polyol comprising 3 to 6 hydroxyl groups;
   of 5 to 40% by weight, with respect to the total weight of the polycondensate, of at least one saturated or unsaturated, linear, branched and/or cyclic, nonaromatic monocarboxylic acid comprising 6 to 32 carbon atoms;
   of 10 to 55% by weight, with respect to the total weight of the polycondensate, of at least one aromatic monocarboxylic acid comprising 7 to 11 carbon atoms, optionally in addition substituted by 1 to 3 saturated or unsaturated, linear, branched and/or cyclic, alkyl radicals which comprise 1 to 32 carbon atoms;
   of 10 to 25% by weight, with respect to the total weight of the polycondensate, of at least one saturated or unsaturated, indeed even aromatic, linear, branched and/or cyclic, polycarboxylic acid comprising at least 2 COOH carboxyl groups, in particular 2 to 4 COOH groups; and/or a cyclic anhydride of such a polycarboxylic acid,
b) at least one compound chosen from:
   block copolymers of following formula (I):

HO—(CH₂—CH₂—O)ₙ—(CₓH₂ₓO)ₘ—(CH₂—CH₂—O)ₚ—H,
in which n, m and p are independently integers ranging from 1 to 1000 and x is an integer strictly greater than 2 and less than or equal to 10,
copolymers comprising at least one polyethylene glycol block and at least one polypropylene glycol block, the said copolymer exhibiting a molecular weight of greater than or equal to 1000 g/mol, and their mixtures.

The combination of the polyoxyalkylene block copolymer and of the polycondensate of alkyd type makes it possible to formulate nail varnish compositions which make it possible to obtain, on the nails, a film of improved gloss with respect to the state of the art while exhibiting a satisfactory homogeneity and good hold.

The composition according to the invention comprises a cosmetically acceptable medium, that is to say a non-toxic medium capable of being applied to the skin, superficial surface growths or lips of the human face.

Another subject-matter of the invention is a cosmetic method for making up or for the non-therapeutic care of the nails, comprising the application, to the nails, of at least one layer of a nail varnish composition as defined above.

A further subject-matter of the invention is the use of the said composition for obtaining, after application to the nails, a glossy varnish film.

### 1) Polycondensate

The polycondensate present in the composition according to the invention is capable of being obtained by reaction:
of 15 to 30% by weight, with respect to the total weight of the polycondensate, of at least one polyol comprising 3 to 6 hydroxyl groups;
of 5 to 40% by weight, with respect to the total weight of the polycondensate, of at least one saturated, linear, branched and/or cyclic, nonaromatic monocarboxylic acid comprising 6 to 32 carbon atoms;
of 10 to 55% by weight, with respect to the total weight of the polycondensate, of at least one aromatic monocarboxylic acid comprising 7 to 11 carbon atoms, optionally in addition substituted by 1 to 3 saturated or unsaturated, linear, branched and/or cyclic, alkyl radicals which comprise 1 to 32 carbon atoms;
of 10 to 25% by weight, with respect to the total weight of the polycondensate, of at least one saturated or unsaturated, indeed even aromatic, linear, branched and/or cyclic, polycarboxylic acid comprising at least 2 COOH carboxyl groups, in particular 2 to 4 COOH groups; and/or a cyclic anhydride of such a polycarboxylic acid.

The polycondensates of the composition according to the invention are advantageously branched; it may be supposed that this makes it possible to generate a network by entanglement of the polymer chains and thus to obtain the desired properties, in particular in terms of improved hold and in terms of solubility. This is because it has been found that linear polycondensates do not make it possible to obtain a significant improvement in the hold of the composition and that polycondensates of dendrimer type, the chains of which are uniform, do not exhibit an optimum solubility.

The polycondensate can be present in the composition according to the invention in a content as dry matter ranging from 0.5 to 50% by weight, preferably from 0.5 to 40% by weight, in particular from 1 to 25% by weight and better still from 5 to 20% by weight, with respect to the weight of the final composition.

The polycondensates according to the invention are polycondensates of alkyd type and are thus capable of being obtained by esterification/polycondensation of the constituents described below according to methods known to a person skilled in the art.

One of the constituents necessary for the preparation of the polycondensates according to the invention is a compound comprising 3 to 6 hydroxyl groups (polyol), in particular 3 to 4 hydroxyl groups. Use may very obviously be made of a mixture of such polyols. The said polyol can in particular be a saturated or unsaturated, linear, branched and/or cyclic, carbon compound, in particular hydrocarbon compound, comprising 3 to 18 carbon atoms, in particular 3 to 12 carbon atoms, indeed even 4 to 10 carbon atoms, and 3 to 6 hydroxyl (OH) groups which can additionally comprise one or more oxygen atoms intercalated in the chain (ether functional group).

The said polyol is preferably a saturated, linear or branched, hydrocarbon compound comprising 3 to 18 carbon atoms, in particular 3 to 12 carbon atoms, indeed even 4 to 10 carbon atoms, and 3 to 6 hydroxyl (OH) groups.

It can be chosen, alone or as a mixture, from:
triols, such as 1,2,6-hexanetriol, trimethylolethane, trimethylolpropane or glycerol;
tetraols, such as pentaerythritol (tetramethylolmethane), erythritol, diglycerol or ditrimethylolpropane;
pentols, such as xylitol,
hexols, such as sorbitol and mannitol; or also dipentaerythritol or triglycerol.

Preferably, the polyol is chosen from glycerol, pentaerythritol, sorbitol and their mixtures and better still is pentaerythritol.

The polyol or the polyol mixture preferably represents 15 to 30% by weight, in particular 16 to 28% by weight and better still 18 to 25% by weight, of the total weight of the final polycondensate.

Another constituent necessary for the preparation of the polycondensates according to the invention is a saturated or unsaturated, linear, branched and/or cyclic, nonaromatic monocarboxylic acid comprising 6 to 32 carbon atoms, in particular 8 to 28 carbon atoms and better still 10 to 20 carbon atoms, indeed even 12 to 18 carbon atoms. Use may very obviously be made of a mixture of such nonaromatic monocarboxylic acids.

The term “nonaromatic monocarboxylic acid” is understood to mean a compound of formula RCOOH in which R is a saturated or unsaturated, linear, branched and/or cyclic, hydrocarbon radical comprising 5 to 31 carbon atoms, in particular 7 to 27 carbon atoms and better still 9 to 19 carbon atoms, indeed even 11 to 17 carbon atoms.

Preferably, the R radical is saturated. Better still, the said R radical is linear or branched and preferably comprises 5 to 31 carbon atoms.

Mention may be made, among nonaromatic monocarboxylic acids capable of being employed, alone or as a mixture, of:
saturated monocarboxylic acids, such as caproic acid, caprylic acid, isoheptanoic acid, 4-ethylpentanoic acid, 2-ethylhexanoic acid, 4,5-dimethylhexanoic acid, 2-heptylheptanoic acid, 3,5,5-trimethylhexanoic acid, octanoic acid, isooctanoic acid, nonanoic acid, decanoic acid, isononanoic acid, lauric acid, tridecanoic acid, myristic acid, palmitic acid, stearic acid, isostearic acid, arachidic acid, behenic acid, cerotic (hexacosanoic) acid, cyclopentanecarboxylic acid, cyclopentaneacetic acid, 3-cyclopentylpropionic acid, cyclohexanecarboxylic acid, cyclohexylacetic acid or 4-cyclohexylbutyric acid;
unsaturated but nonaromatic monocarboxylic acids, such as caproleic acid, undecylenic acid, dodecylenic acid, myristoleic acid, palmitoleic acid, oleic acid, elaidic acid, gondoic acid or erucic acid.

Use is preferably made of 2-ethylhexanoic acid, isooctanoic acid, lauric acid, palmitic acid, isostearic acid and their mixtures and better still isostearic acid alone.

The said nonaromatic monocarboxylic acid or the mixture of the said acids preferably represents 5 to 40% by weight, in particular 8 to 38% by weight and better still 10 to 35% by weight, of the total weight of the final polycondensate.

Another constituent necessary for the preparation of the polycondensates according to the invention is an aromatic monocarboxylic acid comprising 7 to 11 carbon atoms, optionally in addition substituted by 1 to 3 saturated or unsaturated, linear, branched and/or cyclic, alkyl radicals which comprise 1 to 32 carbon atoms, in particular 2 to 12 carbon atoms, indeed even 3 to 8 carbon atoms.

Use may very obviously be made of a mixture of such aromatic monocarboxylic acids.

The term “aromatic monocarboxylic acid” is understood to mean a compound of formula R′COOH in which R′ is an aromatic hydrocarbon radical comprising 6 to 10 carbon atoms and in particular the benzoic and naphthoic radicals.

The said R′ radical can in addition be substituted by 1 to 3 saturated or unsaturated, linear, branched and/or cyclic, alkyl radicals comprising 1 to 32 carbon atoms, in particular 2 to 12 carbon atoms, indeed even 3 to 8 carbon atoms, chosen in particular from methyl, ethyl, propyl, isopropyl, butyl, isobutyl, tert-butyl, pentyl, isopentyl, neopentyl, cyclopentyl, hexyl, cyclohexyl, heptyl, isoheptyl, octyl or isooctyl.

Mention may be made, among aromatic monocarboxylic acids capable of being employed, alone or as a mixture, of benzoic acid, o-toluic acid, m-toluic acid, p-toluic acid, 1-naphthoic acid, 2-naphthoic acid, 4-(tert-butyl)benzoic acid, 1-methyl-2-naphthoic acid or 2-isopropyl-1-naphthoic acid.

Use may preferably be made of benzoic acid, o-toluic acid, m-toluic acid or 1-naphthoic acid, alone or as mixtures, and better still benzoic acid alone.

The said aromatic monocarboxylic acid or the mixture of the said acids preferably represents 10 to 55% by weight, in particular 20 to 52% by weight, indeed even 22 to 52% by weight and better still 25 to 50% by weight, of the total weight of the final polycondensate.

Another constituent necessary for the preparation of the polycondensates according to the invention is a saturated or unsaturated, indeed even aromatic, linear, branched and/or cyclic, polycarboxylic acid comprising at least 2 COOH carboxyl groups, in particular 2 to 4 COOH groups, and/or a cyclic anhydride of such a polycarboxylic acid. Use may very clearly be made of a mixture of such polycarboxylic acids and/or anhydrides.

The said polycarboxylic acid can be chosen in particular from saturated or unsaturated, indeed even aromatic, linear, branched and/or cyclic, polycarboxylic acids comprising 2 to 20 carbon atoms, in particular 3 to 18 carbon atoms and better still 4 to 12 carbon atoms, indeed even 4 to 10 carbon atoms; the said acid comprises at least two COOH carboxyl groups, preferably from 2 to 4 COOH groups.

Preferably, the said polycarboxylic acid is saturated linear aliphatic and comprises 2 to 20 carbon atoms, in particular 3 to 18 carbon atoms, indeed even 4 to 12 carbon atoms, or else is aromatic and comprises 8 to 12 carbon atoms. It preferably comprises 2 to 4 COOH groups.

The said cyclic anhydride of such a polycarboxylic acid can correspond in particular to one of the following formulae:

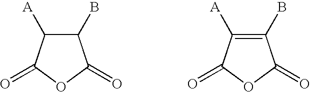

in which the A and B groups are, independently of one another:
a hydrogen atom,
a saturated or unsaturated, linear, branched and/or cyclic, aliphatic carbon radical or else an aromatic radical comprising 1 to 16 carbon atoms, in particular 2 to 10 carbon atoms, indeed even 4 to 8 carbon atoms, in particular methyl or ethyl;
or else A and B, taken together, form a saturated or unsaturated, indeed even aromatic, ring comprising, in total, 5 to 7 carbon atoms and in particular 6 carbon atoms.

Preferably, A and B represent a hydrogen atom or together form an aromatic ring comprising a total of 6 carbon atoms.

Mention may be made, among polycarboxylic acids or their anhydrides capable of being used, alone or as a mixture, of:
dicarboxylic acids, such as decanedioic acid, dodecanedioic acid, cyclopropanedicarboxylic acid, cyclohexanedicarboxylic acid, cyclobutanedicarboxylic acid, naphthalene-1,4-dicarboxylic acid, naphthalene-2,3-dicarboxylic acid, naphthalene-2,6-dicarboxylic acid, suberic acid, oxalic acid, malonic acid, succinic acid, phthalic acid, terephthalic acid, isophthalic acid, pimelic acid, sebacic acid, azelaic acid, glutaric acid, adipic acid, fumaric acid or maleic acid;
tricarboxylic acids, such as cyclohexanetricarboxylic acid, trimellitic acid, 1,2,3-benzenetricarboxylic acid or 1,3,5-benzenetricarboxylic acid;
tetracarboxylic acids, such as butanetetracarboxylic acid and pyromellitic acid;
cyclic anhydrides of these acids and in particular phthalic anhydride, trimellitic anhydride, maleic anhydride and succinic anhydride.

Use may preferably be made of phthalic anhydride and/or isophthalic acid and better still isophthalic acid alone.

The said polycarboxylic acid and/or its cyclic anhydride preferably represents 10 to 25% by weight, in particular 11 to 22% by weight and better still 12 to 20% by weight, of the total weight of the final polycondensate.

The polycondensate according to the invention can additionally comprise a silicone possessing a hydroxyl (OH) and/or carboxyl (COOH) functional group.

It can comprise 1 to 3 hydroxyl and/or carboxyl functional groups and preferably comprises two hydroxyl functional groups or else two carboxyl functional groups.

These functional groups can be situated at the chain end or in the chain but advantageously at the chain end.

Use is preferably made of silicones having a weight-average molecular weight (Mw) of between 300 and 20 000, in particular 400 and 10 000, indeed even 800 and 4000.

This silicone can be of formula:

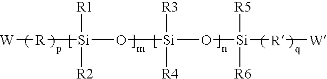

in which:
W and W′ are, independently of one another, OH or COOH; preferably, W═W′;
p and q are, independently of one another, equal to 0 or 1,
R are R′ are, independently of one another, a saturated or unsaturated, indeed even aromatic, linear, branched and/or cyclic, divalent carbon radical, in particular hydrocarbon radical, comprising 1 to 12 carbon atoms, in particular 2 to 8 carbon atoms, and optionally comprising, in addition, one or more heteroatoms chosen from O, S and N, in particular O (ether);
   in particular, R and/or R′ can be of formula —(CH₂)ₐ— with a=1-12, and in particular methylene, ethylene, propylene or phenylene;
   or else of formula —[(CH₂)ₓO]_{z}— with x=1, 2 or 3 and z=1-10; in particular, x=2 or 3 and z=1-4; and better still x=3 and z=1.
R1 to R6 are, independently of one another, a saturated or unsaturated, indeed even aromatic, linear, branched and/or cyclic, carbon radical comprising 1 to 20 carbon atoms, in particular 2 to 12 carbon atoms; preferably, R1 to R6 are saturated or else aromatic and can in particular be chosen from alkyl radicals, in particular the methyl, ethyl, propyl, isopropyl, butyl, pentyl, hexyl, octyl, decyl, dodecyl and octadecyl radicals, cycloalkyl radicals, in particular the cyclohexyl radical, aryl radicals, in particular phenyl and naphthyl, arylalkyl radicals, in particular benzyl and phenylethyl, and also the tolyl and xylyl radicals.
m and n are, independently of one another, integers between 1 and 140 and are such that the weight-average molecular weight (Mw) of the silicone is between 300 and 20 000, in particular 400 and 10 000, indeed even between 800 and 4000.

Mention may in particular be made of α,ω-diol or α,ω-dicarboxyl polyalkylsiloxanes and in particular α,ω-diol polydimethylsiloxanes and α,ω-dicarboxyl polydimethylsiloxanes; α,ω-diol or α,ω-dicarboxyl polyarylsiloxanes and in particular α,ω-diol or α,ω-dicarboxyl polyphenylsiloxanes; polyarylsiloxanes possessing silanol functional groups, such as polyphenylsiloxane; polyalkylsiloxanes possessing silanol functional groups, such as polydimethylsiloxane; polyaryl/alkylsiloxanes possessing silanol functional groups, such as polyphenyl/methylsiloxane or polyphenyl/propylsiloxane. Use will very particularly be made of α,ω-diol polydimethylsiloxanes with a weight-average molecular weight (Mw) of between 400 and 10 000, indeed even between 500 and 5000 and in particular between 800 and 4000.

When it is present, the said silicone can preferably represent 0.1 to 15% by weight, in particular 1 to 10% by weight, indeed even 2 to 8% by weight, of the weight of the polycondensate.

In a preferred embodiment of the invention, the aromatic monocarboxylic acid is present in a molar amount greater than or equal to that of the nonaromatic monocarboxylic acid; in particular, the ratio of the number of moles of aromatic monocarboxylic acid to the number of moles of nonaromatic monocarboxylic acid is preferably between 1.2 and 8, in particular between 1.3 and 7.8, indeed even between 1.4 and 7.5 and better still between 1.9 and 7.

It has been found that this makes it possible in particular to obtain a polymer which is advantageously soluble in the “short” esters (butyl or ethyl acetate type) generally employed to formulate cosmetic compositions of nail varnish type; furthermore, the film obtained exhibits a stiffness suitable for the use thereof in nail varnish formulations.

Preferably, the polycondensate according to the invention is capable of being obtained by reaction:
of at least one polyol chosen, alone or as a mixture, from 1,2,6-hexanetriol, trimethylolethane, trimethylolpropane, glycerol, pentaerythritol, erythritol, diglycerol, ditrimethylolpropane, xylitol, sorbitol, mannitol, dipentaerythritol and/or triglycerol;
   preferably present in an amount of 15 to 30% by weight, in particular 16 to 28% by weight and better still 18 to 25% by weight, with respect to the total weight of the final polycondensate;
of at least one nonaromatic monocarboxylic acid chosen, alone or as a mixture, from caproic acid, caprylic acid, isoheptanoic acid, 4-ethylpentanoic acid, 2-ethylhexanoic acid, 4,5-dimethylhexanoic acid, 2-heptylheptanoic acid, 3,5,5-trimethylhexanoic acid, octanoic acid, isooctanoic acid, nonanoic acid, decanoic acid, isononanoic acid, lauric acid, tridecanoic acid, myristic acid, palmitic acid, stearic acid, isostearic acid, arachidic acid, behenic acid, cerotic (hexacosanoic) acid; cyclopentanecarboxylic acid, cyclopentaneacetic acid, 3-cyclopentylpropionic acid, cyclohexanecarboxylic acid, cyclohexylacetic acid or 4-cyclohexylbutyric acid;
   preferably present in an amount of 5 to 40% by weight, in particular 8 to 38% by weight and better still 10 to 35% by weight, with respect to the total weight of the final polycondensate;
of at least one aromatic monocarboxylic acid chosen, alone or as a mixture, from benzoic acid, o-toluic acid, m-toluic acid, p-toluic acid, 1-naphthoic acid, 2-naphthoic acid, 4-(tert-butyl)benzoic acid, 1-methyl-2-naphthoic acid or 2-isopropyl-1-naphthoic acid; preferably present in an amount of 10 to 55% by weight, in particular 20 to 52% by weight and better still 25 to 50% by weight, with respect to the total weight of the final polycondensate; and
of at least one polycarboxylic acid or one of its anhydrides chosen, alone or as a mixture, from decanedioic acid, dodecanedioic acid, cyclopropanedicarboxylic acid, cyclohexanedicarboxylic acid, cyclobutanedicarboxylic acid, naphthalene-1,4-dicarboxylic acid, naphthalene-2,3-dicarboxylic acid, naphthalene-2,6-dicarboxylic acid, suberic acid, oxalic acid, malonic acid, succinic acid, phthalic acid, terephthalic acid, isophthalic acid, pimelic acid, sebacic acid, azelaic acid, glutaric acid, adipic acid, fumaric acid, maleic acid, cyclohexanetricarboxylic acid, trimellitic acid, 1,2,3-benzenetricarboxylic acid, 1,3,5-benzenetricarboxylic acid, butanetetracarboxylic acid, pyromellitic acid, phthalic anhydride, trimellitic anhydride, maleic anhydride and succinic anhydride;
   preferably present in an amount of 10 to 25% by weight, in particular 11 to 22% by weight and better still 12 to 20% by weight, with respect to the total weight of the final polycondensate.

Preferably, the polycondensate according to the invention is capable of being obtained by reaction:
of at least one polyol chosen, alone or as a mixture, from glycerol, pentaerythritol, sorbitol and their mixtures and better still pentaerythritol alone, present in an amount of 15 to 30% by weight, in particular 16 to 28% by weight and better still 18 to 25% by weight, with respect to the total weight of the final polycondensate;
of at least one nonaromatic monocarboxylic acid chosen, alone or as a mixture, from 2-ethylhexanoic acid, isooctanoic acid, lauric acid, palmitic acid, isostearic acid and their mixtures and better still isostearic acid alone, present in an amount of 5 to 40% by weight, in particular 8 to 38% by weight and better still 10 to 35% by weight, with respect to the total weight of the final polycondensate;
of at least one aromatic monocarboxylic acid chosen, alone or as a mixture, from benzoic acid, o-toluic acid, m-toluic acid, 1-naphthoic acid and better still benzoic acid alone, present in an amount of 10 to 55% by weight, in particular 20 to 52% by weight and better still 25 to 50% by weight, with respect to the total weight of the final polycondensate; and
of at least one polycarboxylic acid or one of its anhydrides chosen, alone or as a mixture, from phthalic anhydride and isophthalic acid and better still isophthalic acid alone, present in an amount of 10 to 25% by weight, in particular 11 to 22% by weight and better still 12 to 20% by weight, with respect to the total weight of the final polycondensate.

Preferably, the polycondensate according to the invention exhibits:
an acid number, expressed as mg of potassium hydroxide per g of polycondensate, of greater than or equal to 8, in particular of between 8 and 40 and better still of between 10 and 30; and/or
a hydroxyl number, expressed as mg of potassium hydroxide per g of polycondensate, of greater than or equal to 30, in particular of between 30 and 100 and better still of between 40 and 90.

These acid and hydroxyl numbers can be easily determined by a person skilled in the art by the usual analytical methods.

Preferably, the polycondensate according to the invention exhibits a viscosity, measured at 110° C., of between 75 and 6000 mPa·s, in particular between 80 and 5500 mPa·s, indeed even between 90 and 5000 mPa·s and better still between 200 and 4800 mPa·s. This viscosity is measured in the way described before the examples.

Furthermore, the polycondensate is advantageously soluble in short esters comprising a total of 3 to 8 carbon atoms, in particular the acetates of C₁-C₆ carboxylic acids and in particular butyl acetate and/or ethyl acetate.

The term “soluble” is understood to mean that the polymer forms a clear solution in butyl acetate or ethyl acetate at 25° C. in a proportion of at least 50% by weight; preferably, the polymer according to the invention is soluble in a proportion of at least 70% by weight in butyl acetate or ethyl acetate.

Preferably, the solution of the polymer according to the invention in butyl acetate or ethyl acetate at 25° C. at a concentration of 70% by weight exhibits a viscosity of between 100 and 1500 mPa·s, in particular between 120 and 900 mPa·s. The measurement method is given before the examples.

The polycondensate according to the invention can be prepared by the esterification/polycondensation processes normally employed by a person skilled in the art. By way of illustration, a general preparation process consists:
in mixing the polyol and the aromatic and nonaromatic monocarboxylic acids,
in heating the mixture under an inert atmosphere, first up to the melting point (generally 100-130° C.) and subsequently at a temperature of between 150 and 220° C. until the monocarboxylic acids have been completely consumed (achieved when the acid number is less than or equal to 1), preferably while distilling off the water formed as it is formed, then
in optionally cooling the mixture to a temperature of between 90 and 150° C.,
in adding the polycarboxylic acid and/or the cyclic anhydride and optionally the silicone possessing hydroxyl or carboxyl functional groups, all at once or sequentially, then
in again heating at a temperature of less than or equal to 220° C., in particular of between 170 and 220° C., preferably while continuing to remove the water formed, until the required characteristics in terms of acid number, of viscosity, of hydroxyl number and of solubility are obtained.

It is possible to add conventional esterification catalysts, for example of sulphonic acid type (in particular at a concentration by weight of between 1 and 10%) or titanate type (in particular at a concentration by weight of between 5 and 100 ppm).

It is also possible to carry out the reaction, in all or part, in an inert solvent, such as xylene, and/or under reduced pressure, in order to facilitate the removal of the water.

Advantageously, neither catalyst nor solvent is used.

The said preparation process can additionally comprise a stage of addition of at least one antioxidizing agent to the reaction medium, in particular at a concentration by weight of between 0.01 and 1%, with respect to the total weight of monomers, so as to limit possible decompositions relating to prolonged heating. The antioxidizing agent can be of primary type or of secondary type and can be chosen from hindered phenols, aromatic secondary amines, organophosphorus compounds, sulphur compounds, lactones, acrylate bisphenols; and their mixtures.

Mention may in particular be made, among the antioxidants which are particularly preferred, of BHT, BHA, TBHQ, 1,3,5-trimethyl-2,4,6-tris(3,5-di(tert-butyl)-4-hydroxybenzyl)benzene, octadecyl 3,5-di(tert-butyl)-4-hydroxycinnamate, methanetetrakis[methylene-3-(3,5-di(tert-butyl)-4-hydroxyphenyl)propionate], octadecyl 3-(3,5-di(tert-butyl)-4-hydroxyphenyl)-propionate, 2,5-di(tert-butyl)hydroquinone, 2,2-methylenebis(4-methyl-6-(tert-butyl)phenol), 2,2-methylenebis(4-ethyl-6-(tert-butyl)phenol), 4,4-butylidenebis(6-(tert-butyl)-m-cresol), N,N′-hexamethylene bis(3,5-di(tert-butyl)-4-hydroxyhydro-cinnamamide), pentaerythritol tetrakis(3-(3,5-di(tert-butyl)-4-hydroxyphenyl)propionate), in particular that sold by Ciba under the name Irganox 1010, octadecyl 3-(3,5-di(tert-butyl)-4-hydroxphenyl) propionate, in particular that sold by Ciba under the name Irganox 1076, 1,3,5-tris(3,5-di(tert-butyl)-4-hydroxybenzyl)-1,3,5-triazine-2,4,6(1H,3H,5H)-trione, in particular that sold by Mayzo of Norcross, Ga., under the name BNX 3114, di(stearyl) pentaerythritol diphosphite, tris(2,4-di(tert-butyl)phenyl) phosphate, in particular that sold by Ciba under the name Irgafos 168, dilauryl thiodipropionate, in particular that sold by Ciba under the name Irganox PS800, bis(2,4-di(tert-butyl)phenyl) pentaerythritol diphosphite, in particular that sold by Ciba under the name Irgafos 126, bis(2,4-bis[2-phenylpropan-2-yl]phenyl)pentaerythritol diphosphite, triphenyl phosphite, (2,4-di(tert-butyl)phenyl) pentaerythritol diphosphite, in particular that sold by GE Specialty Chemicals under the name Ultranox 626, tris(nonylphenyl) phosphite, in particular that sold by Ciba under the name Irgafos TNPP, the 1:1 mixture of N,N′-hexamethylene bis(3,5-di(tert-butyl)-4-hydroxy-hydrocinnamamide) and of tris(2,4-di(tert-butyl)phenyl) phosphate, in particular that sold by Ciba under the name Irganox B 1171, tetrakis(2,4-di(tert-butyl)phenyl) phosphite, in particular that sold by Ciba under the name Irgafos P-EPQ, distearyl thiodipropionate, in particular that sold by Ciba under the name Irganox PS802, 2,4-bis(octylthiomethyl)-o-cresol, in particular that sold by Ciba under the name Irganox 1520, 4,6-bis(dodecylthiomethyl)-o-cresol, in particular that sold by Ciba under the name Irganox 1726.

### 2) Polyoxyalkylene Block Copolymer

The term “block copolymer” is understood to mean a polymer comprising at least 2 distinct blocks or sequences, preferably at least 3 distinct sequences.

The block copolymer of the composition according to the invention can be chosen from the compounds of the following formula (I):

HO—(CH₂—CH₂—O)ₙ—(CₓH₂ₓO)ₘ—(CH₂—CH₂—O)ₚ—H,
in which n, m and p are independently integers ranging from 1 to 1000 and x is an integer strictly greater than 2 and less than or equal to 10.

According to one embodiment, the block copolymer comprises at least one polyethylene glycol block and at least one polypropylene glycol block.

It can then correspond to the following formula:

HO—(CH₂—CH₂—O)ₙ—(CₓH₂ₓO)ₘ—H
with n and m integers between 1 and 1000 and x an integer strictly greater than 2 and less than 10.

The block copolymer of the composition advantageously exhibits a molecular weight of greater than or equal to 1000 g/mol, preferably of greater than or equal to 1500 g/mol, better still of greater than 2000 g/mol.

Mention may be made, as copolymers which can be used in the composition according to the invention, of copolymers of propylene oxide and of ethylene oxide, also known as EO/PO polycondensates, such as, for example, the polyethylene glycol/polypropylene glycol/polyethylene glycol triblock polycondensates sold under the “Synperonic” names, such as Synperonic PE/L44, Synperonic PE/L64 (13EO/13PO/13EO) with an MW of 2900 and Synperonic PE/F127 by ICI, and their mixtures.

The polyoxyalkylene block copolymer or copolymers can be present in an amount as dry matter ranging from 0.1 to 15% by weight, with respect to the total weight of the composition, preferably from 0.5 to 10% by weight and better still from 1 to 8% by weight.

### Organic Solvent Medium

The composition according to the invention comprises an organic solvent medium comprising an organic solvent or a mixture of organic solvents.

The organic solvent can be chosen from:
ketones which are liquid at ambient temperature, such as methyl ethyl ketone, methyl isobutyl ketone, diisobutyl ketone, isophorone, cyclohexanone or acetone;
alcohols which are liquid at ambient temperature, such as ethanol, isopropanol, diacetone alcohol, 2-butoxyethanol or cyclohexanol;
propylene glycol ethers which are liquid at ambient temperature, such as propylene glycol monomethyl ether, propylene glycol monomethyl ether acetate or dipropylene glycol mono(n-butyl)ether;
cyclic ethers, such as γ-butyrolactone;
short-chain esters (having from 3 to 8 carbon atoms in total), such as ethyl acetate, methyl acetate, propyl acetate, isopropyl acetate, n-butyl acetate, isopentyl acetate, methoxypropyl acetate or butyl lactate;
ethers which are liquid at ambient temperature, such as diethyl ether, dimethyl ether or dichlorodiethyl ether;
alkanes which are liquid at ambient temperature, such as decane, heptane, dodecane or cyclohexane;
alkyl sulphoxides, such as dimethyl sulphoxide;
aldehydes which are liquid at ambient temperature, such as benzaldehyde or acetaldehyde;
ethyl 3-ethoxypropionate;
carbonates, such as propylene carbonate or dimethyl carbonate;
acetals, such as methylal;
and their mixtures.

Preferably, the solvent is chosen from short-chain esters having from 3 to 8 carbon atoms in total, such as ethyl acetate, methyl acetate, propyl acetate, isopropyl acetate, n-butyl acetate, isopentyl acetate, methoxypropyl acetate, butyl lactate and their mixtures.

The organic solvent medium can represent from 10 to 95% by weight, with respect to the total weight of the composition, preferably from 15% to 80% by weight and better still from 60 to 80% by weight.

The composition according to the invention can optionally comprise an aqueous medium; in this case, the aqueous medium is present in a content of less than or equal to 2% by weight, with respect to the total weight of the composition, preferably of less than or equal to 1% by weight.

### Film-Forming Polymer

The composition can advantageously comprise a film-forming polymer other than the polycondensate of alkyd type described above.

According to the present invention, the term “film-forming polymer” is understood to mean a polymer capable of forming, by itself alone or in the presence of an additional agent which is able to form a film, a continuous film which adheres to a support, in particular to keratinous substances.

Mention may be made, among film-forming polymers which can be used in the composition of the present invention, of synthetic polymers of radical type or of polycondensate type, polymers of natural origin and their mixtures.

The film-forming polymer can be chosen in particular from cellulose polymers, such as nitrocellulose, cellulose acetate, cellulose acetate butyrate, cellulose acetate propionate or ethyl cellulose, or alternatively polyurethanes, acrylic polymers, vinyl polymers, polyvinyl butyrals, alkyd resins, polyester resins, for example resulting from the polycondensation of adipic acid, of a polyalcohol preferably comprising from 2 to 6 carbon atoms and a compound comprising three acid and/or anhydride functional groups and preferably having from 8 to 16 carbon atoms, resins resulting from aldehyde condensation products, such as hydrogenated ketone/formaldehyde resins, arylsulphonamide-formaldehyde resins, for example toluenesulphonamide-formaldehyde resin, arylsulphonamide-epoxy resins or ethyltosylamide resins.

Use may in particular be made, as film-forming polymer, of nitrocellulose RS ⅛ sec.; RS ¼ sec.; RS ½ sec.; RS 5 sec.; RS 15 sec.; RS 35 sec.; RS 75 sec.; RS 150 sec.; AS ¼ sec.; AS ½ sec.; SS ¼ sec.; SS ½ sec.; SS 5 sec., sold in particular by Hercules; polyester resins with the CTFA name “Adipic Acid/Neopentyl Glycol/Trimellitic Anhydride Copolymer”, such as Uniplex 670-P from Unitex Chemical Corporation, a hydrogenated ketone/formaldehyde resin, such as that sold under the name Synthetic Resin SK by Degussa, the toluenesulphonamide-formaldehyde resins “Ketjentflex MS80” from Akzo or “Santolite MHP” or “Santolite MS 80” from Faconnier or “Resimpol 80” from Pan Americana, the alkyd resin “Beckosol ODE 230-70-E” from Dainippon, the acrylic resin “Acryloid B66” from Röhm & Haas, or the polyurethane resin “Trixene PR 4127” from Baxenden.

According to one embodiment of the invention, the film-forming polymer is a film-forming linear ethylenic sequential polymer which preferably comprises at least one first sequence and at least one second sequence having different glass transition temperature (Tg), the said first and second sequences being connected to one another via an intermediate sequence comprising at least one constituent monomer of the first sequence and at least one constituent monomer of the second sequence.

Advantageously, the first and second sequences of the sequential polymer are incompatible with one another. Such polymers are described, for example, in the documents EP 1411069 or WO 04/028488.

The film-forming polymer(s) can be present in the composition according to the invention in a total content as dry matter ranging from 0.1% to 60% by weight, with respect to the total weight of the composition, preferably from 2% to 50% by weight and better still from 5% to 30% by weight.

Additional Agent which is Able to Form a Film

An additional agent which is able to form a film can be provided in order to improve the film-forming properties of the nail varnish composition.

Such an additional agent which is able to form a film can be chosen from all the compounds known to a person skilled in the art as being capable of fulfilling the desired role and can in particular be chosen from plasticizing agents and coalescence agents for the film-forming polymer(s).

Thus, the composition can additionally comprise at least one plasticizing agent and/or one coalescence agent. Mention may in particular be made, alone or as a mixture, of conventional plasticizers and coalescence agents, such as:
glycols and their derivatives, such as diethylene glycol ethyl ether, diethylene glycol methyl ether, diethylene glycol butyl ether or diethylene glycol hexyl ether, ethylene glycol ethyl ether, ethylene glycol butyl ether or ethylene glycol hexyl ether;
polyethylene glycols, polypropylene glycols, polyethylene glycol-polypropylene glycol copolymers other than the polyoxyalkylene copolymer described above and their mixtures, in particular polypropylene glycols of high molecular weight, for example having a molecular weight ranging from 500 to 15,000, such as, for example
glycol esters;
propylene glycol derivatives and in particular propylene glycol phenyl ether, propylene glycol diacetate, dipropylene glycol ethyl ether, tripropylene glycol methyl ether and diethylene glycol methyl ether, or propylene glycol butyl ether;
acid esters, in particular carboxylic acid esters, such as citrates, phthalates, adipates, carbonates, tartrates, phosphates or sebacates;
esters resulting from the reaction of a monocarboxylic acid of formula R₁₁COOH with a diol of formula HOR₁₂OH with R₁₁ and R₁₂, which are identical or different, representing a saturated or unsaturated, linear, branched or cyclic, hydrocarbon chain preferably comprising from 3 to 15 carbon atoms and optionally comprising one or more heteroatoms, such as N, O or S, in particular the monoester resulting from the reaction of isobutyric acid and of octanediol, such as 2,2,4-trimethylpentane-1,3-diol, such as that sold under the reference Texanol Ester Alcohol by Eastman Chemical;
oxyethylenated derivatives, such as oxyethylenated oils, in particular vegetable oils, such as castor oil;
dimethicone copolyols, in particular possessing α,ω propyl polyoxypropylene groups, and
their mixtures.

The additional agent which is able to form a film can represent from 0.01 to 40% by weight, preferably from 0.05 to 30% by weight and better still from 0.1 to 20% by weight, with respect to the total weight of the composition.

### Thickening Agent

The composition can comprise, apart from the block copolymer, a thickening agent which can in particular be chosen from: silicas, in particular hydrophobic silicas, such as those described in the document EP-A-898 960, for example sold under the references “Aerosil R812®” by Degussa, “Cab-O-Sil TS-530®”, “Cab-O-Sil TS-610®” or “Cab-O-Sil TS-720®” by Cabot or “Aerosil R972®”, “Aerosil R974®” by Degussa; clays, such as montmorillonite; modified clays, such as bentones, for example stearalkonium hectorite or stearalkonium bentonite; polysaccharide alkyl ethers (in particular for which the alkyl group comprises from 1 to 24 carbon atoms, preferably from 1 to 10, better still from 1 to 6 and more especially from 1 to 3), such as those described in the document EP-A-898 958.

The total proportion of thickening agent(s) in the compositions according to the invention can range from 0.01 to 15% by weight, with respect to the total weight of the composition, preferably from 0.05 to 10% by weight and better still from 0.1 to 5% by weight.

According to a specific embodiment, the composition comprises a thickening agent chosen from bentones in a content of less than or equal to 3% by weight, with respect to the total weight of the composition.

### Colouring Material

The composition according to the invention can additionally comprise one or more colouring materials chosen from water-soluble dyes and pulverulent colouring materials, such as pigments, pearlescent agents and glitter, well known to a person skilled in the art. The colouring materials can be present in the composition in a content ranging from 0.01% to 50% by weight, with respect to the weight of the composition, preferably from 0.01% to 30% by weight.

The term “pigments” should be understood as meaning white or coloured and inorganic or organic particles of any shape which are insoluble in the physiological medium and which are intended to colour the composition.

The term “pearlescent agents” should be understood as meaning iridescent particles of any shape produced in particular by certain molluscs in their shells or else synthesized.

The pigments can be white or coloured and inorganic or organic. Mention may be made, among inorganic pigments, of titanium dioxide, optionally surface-treated, zirconium or cerium oxides, and also zinc, iron (black, yellow or red) or chromium oxides, manganese violet, ultramarine blue, chromium hydrate and ferric blue, or metal powders, such as aluminium powder or copper powder.

Mention may be made, among organic pigments, of carbon black, pigments of D & C type, and lakes, based on cochineal carmine, of barium, strontium, calcium or aluminium.

The pearlescent pigments can be chosen from white pearlescent pigments, such as mica covered with titanium oxide or with bismuth oxychloride, coloured pearlescent pigments, such as titanium oxide-coated mica covered with iron oxides, titanium oxide-coated mica covered with in particular ferric blue or chromium oxide, or titanium oxide-coated mica covered with an organic pigment of the abovementioned type, and pearlescent pigments based on bismuth oxychloride. The water-soluble dyes are, for example, beetroot juice or methylene blue.

The composition according to the invention can additionally comprise one or more fillers, in particular in a content ranging from 0.01% to 50% by weight, with respect to the total weight of the composition, preferably ranging from 0.01% to 30% by weight. The term “fillers” should be understood as meaning colourless or white and inorganic or synthetic particles of any shape which are insoluble in the medium of the composition, whatever the temperature at which the composition is manufactured. These fillers are used in particular to modify the rheology or the texture of the composition.

The fillers can be inorganic or organic fillers of any shape, platelet, spherical or oblong, whatever the crystallographic form (for example, sheet, cubic, hexagonal, orthorhombic, and the like). Mention may be made of talc, mica, silica, kaolin, polyamide (Nylon®) powders (Orgasol® from Atochem), poly-β-alanine powders, polyethylene powders, powders formed of tetrafluoroethylene polymers (Teflon®), lauroyllysine, starch, boron nitride, polymeric hollow microspheres, such as those of polyvinylidene chloride/acrylonitrile, for example Expancel® (Nobel Industrie), or of acrylic acid copolymers (Polytrap® from Dow Corning), silicone resin microbeads (Tospearls® from Toshiba, for example), polyorganosiloxane elastomer particles, precipitated calcium carbonate, magnesium carbonate, basic magnesium carbonate, hydroxyapatite, hollow silica microspheres (Silica Beads® from Maprecos), glass or ceramic microcapsules, or metal soaps derived from organic carboxylic acids having from 8 to 22 carbon atoms, preferably from 12 to 18 carbon atoms, for example zinc stearate, magnesium stearate, lithium stearate, zinc laurate or magnesium myristate.

### Other Additives

The composition can additionally comprise other ingredients commonly used in cosmetic compositions. Such ingredients can be chosen from spreading agents, wetting agents, dispersing agents, antifoaming agents, preservatives, UV screening agents, active principles, surfactants, moisturising agents, fragrances, neutralizing agents, stabilizing agents or antioxidants.

Of course, a person skilled in the art will take care to choose this or these optional additional compounds and/or their amounts so that the advantageous properties of the composition for the use according to the invention are not, or not substantially, detrimentally affected by the envisaged addition.

According to another aspect, a subject-matter of the invention is a nail varnish product comprising: i) a container delimiting at least one compartment, the said container being closed by a closure element, and ii) a composition according to the invention, which is positioned inside the said compartment.

The container can have any appropriate form. It can in particular be in the form of a bottle and can be, at least partly, made of a material such as glass. However, materials other than glass may be used, such as thermoplastics, for example PP or PE, or such as a metal.

The closure element can be coupled to the compartment by screwing into the closed position of the container. Alternatively, the coupling between the closure element and the container can be carried out other than by screwing, for example by snapping.

The container is preferably equipped with an applicator which can be in the form of a brush composed of at least one tuft of hairs. Alternatively, the applicator is provided in a form other than a brush, for example in the form of a spatula or of a foam tip.

The examples which follow illustrate the invention without implying limitation. Unless otherwise indicated, the amounts shown are percentages by weight.

### EXAMPLES

### Method for Measuring the Viscosity

a) The viscosity at 110° C. of the polymer is measured using a cone-plate viscometer of Brookfield CAP 1000+ type.

The appropriate cone-plate is determined by a person skilled in the art on the basis of his knowledge; in particular:
between 50 and 500 mPa·s, use may be made of a 02 cone
between 500 and 1000 mPa·s: 03 cone
between 1000 and 4000 mPa·s: 05 cone
between 4000 and 10 000 mPa·s: 06 cone

b) The viscosity at 25° C. of the 70% solution of the polymer in butyl acetate is measured using a Brookfield DV-I viscometer at 30 revolutions/min using an S62 spindle.

### Example 1

### Synthesis of Pentaerythrityl Benzoate/Isophthalate/Isostearate

227.5 g of benzoic acid, 72.8 g of isostearic acid and 118.3 g of pentaerythritol are charged to a reactor equipped with a mechanical stirrer, with an argon inlet and with a distillation system and then gradual heating is carried out, under a gentle argon stream, to 110-130° C. in order to obtain a homogeneous solution. The temperature is then gradually increased up to 180° C. and is maintained for approximately 2 hours. The temperature is again increased up to 220° C. and is maintained until an acid number of less than or equal to 1 is obtained, which takes approximately 18 hours. Cooling is carried out to a temperature of between 100 and 130° C., then 91 g of isophthalic acid are introduced and heating is again gradually carried out up to 220° C. for approximately 11 hours.

430 g of pentaerythrityl benzoate/isophthalate/isostearate polycondensate are thus obtained in the form of a thick oil which solidifies at ambient temperature.

The polycondensate exhibits the following characteristics:
Acid number=12.7
Hydroxyl number=49
η_{110° C.}=25.4 poises (i.e. 2540 mPa·s)
ratio of the number of moles of aromatic monocarboxylic acid to the number of moles of nonaromatic monocarboxylic acid: 7.28.

420 g of polycondensate obtained above are withdrawn and heated to 100-120° C., 180 g of butyl acetate are run in slowly with stirring and then the product is clarified by hot filtration through a sintered glass No. 2.

After cooling to ambient temperature, 600 g of 70% solution of polycondensate in butyl acetate are obtained, which solution exists in the form of a pale yellow viscous liquid having a viscosity at 25° C. of approximately 800 centipoises (mPa·s)

### Example 2

### Nail Varnish

| | |
|---|---|
| 70% Solution as dry matter of the polymer of | 17.8 |
| Example 1 in butyl acetate | |
| Nitrocellulose comprising 30% of isopropyl | 12.9 |
| alcohol (viscosity: E22-1/2 S, Idyl EMV from | |
| Bergerac) | |
| Nitrocellulose comprising 30% of isopropyl | 5.54 |
| alcohol (Idyl E27 from Bergerac) | |
| 70% Adipic acid/trimellitic anhydride/ | 2 |
| neopentyl glycol copolymer in butyl acetate | |
| (“Uniplex 670-P” from Unitex Chemical | |
| Corporation) | |
| Tributyl acetylcitrate | 0.8 |
| 25% Toluenesulphonamide-epoxy resin in butyl | 2.42 |
| acetate | |
| Stearylbenzyldimethylammonium-modified | 2.08 |
| hectorite | |
| N-Ethyl-o,p-toluenesulphonamide | 1.61 |
| Ethylene oxide/propylene oxide/ethylene oxide | 3 |
| (13EO/30PO/13EO) condensate sold by Uniqema | |
| under the reference Synperonic PE/L64 | |
| n-Butyl alcohol | 0.8 |
| n-Propyl acetate | 12.1 |
| Isopropyl alcohol | 0.81 |
| Citric acid monohydrate | 0.09 |
| Ethyl acetate | 19.38 |
| Butyl acetate | q.s. for |
| | 100 |

⁽¹⁾sold by Shell under the trade name Cardura 30 ®.

This varnish, after application to the nails, forms a very glossy transparent film.

### Examples 3 and 4

### Nail Varnishes

A nail varnish according to the invention (Example 3) and a nail varnish according to the prior art (Example 4) were prepared:

| | Example 3 | Example 4 |
|---|---|---|
| 70% Solution as dry matter of the polymer | 17.6 | — |
| of Example 1 in butyl acetate | | |
| Nitrocellulose comprising 30% of | 13.9 | 14.1 |
| isopropyl alcohol (viscosity: E22-1/2 S, | | |
| Idyl EMV from Bergerac) | | |
| Nitrocellulose comprising 30% of | — | 1.99 |
| isopropyl alcohol (viscosity: E24-1/8 S, | | |
| Idyl EMV from Bergerac) | | |
| Nitrocellulose comprising 30% of | — | 1.18 |
| isopropyl alcohol (Azur E80 from | | |
| Bergerac) | | |
| Nitrocellulose comprising 30% of | 1.74 | — |
| isopropyl alcohol (Idyl E27 from | | |
| Bergerac) | | |
| Acetone-formaldehyde resin (SK from | 0.175 | — |
| Degussa) | | |
| 70% Adipic acid/trimellitic anhydride/ | 1.97 | 2 |
| neopentyl glycol copolymer in butyl | | |
| acetate (“Uniplex 670-P” from Unitex | | |
| Chemical Corporation) | | |
| Tributyl acetylcitrate | 3.67 | 0.87 |
| 25% Toluenesulphonamide-epoxy resin in | 2.61 | — |
| butyl acetate | | |
| 70% Phthalic glycerol alkyd resin | — | 13.6 |
| esterified with branched fatty acids in | | |
| ethyl acetate (Cardura 30 ® from Shell) | | |
| Stearylbenzyldimethylammonium-modified | 0.65 | 1.1 |
| hectorite | | |
| N-Ethyl-o,p-toluenesulphonamide | 1.74 | 2.14 |
| Ethylene oxide/propylene oxide/ethylene | 2.95 | — |
| oxide (13EO/30PO/13EO) condensate sold by | | |
| Uniqema under the reference Synperonic | | |
| PE/L64 | | |
| Red 7 lake | 0.2 | 0.2 |
| Polyethylene-coated titanium oxide | 0.02 | 0.02 |
| Red 34 lake | 0.55 | 0.55 |
| Polydimethylsiloxane (viscosity 350 cSt) | — | 0.199 |
| n-Butyl alcohol | 0.87 | — |
| n-Propyl acetate | 13 | — |
| Isopropyl alcohol | 0.25 | 3.22 |
| Citric acid monohydrate | 0.028 | 0.044 |
| Ethyl acetate | 20.9 | 27.18 |
| Butyl acetate | q.s. for | q.s. for |
| | 100 | 100 |

⁽¹⁾sold by Shell under the trade name Cardura 30 ®.

The gloss of each varnish was measured according to the following protocol:

A layer of the composition for which it is desired to evaluate the mean gloss is spread, using an automatic spreader, with a wet thickness of 300 μm over a Leneta brand contrast card with the Form 1A Penopac reference. The layer covers the white background and the black background of the card. Drying is allowed to take place for 24 hours on a stand thermostatically controlled at 30° C. and then the 60° gloss is measured on the white background (3 measurements) and on the black background (3 measurements) using a BYK-Gardner brand glossmeter with the micro-Tri-gloss reference.

The following results are obtained:

| | Example 3 | Example 4 |
|---|---|---|
| 60° Gloss | 90.5 | 84.9 |

The nail varnish according to the invention exhibits a much higher gloss than that of a varnish not comprising the combination of a polycondensate of modified alkyd type and of a polyoxyalkylene copolymer.

1. Nail varnish composition comprising, in an organic solvent medium:
a) at least one polycondensate capable of being obtained by reaction:
of 15 to 30% by weight, with respect to the total weight of the polycondensate, of at least one polyol comprising 3 to 6 hydroxyl groups;
of 5 to 40% by weight, with respect to the total weight of the polycondensate, of at least one saturated or unsaturated, linear, branched and/or cyclic, nonaromatic monocarboxylic acid comprising 6 to 32 carbon atoms;
of 10 to 55% by weight, with respect to the total weight of the polycondensate, of at least one aromatic monocarboxylic acid comprising 7 to 11 carbon atoms, optionally in addition substituted by 1 to 3 saturated or unsaturated, linear, branched and/or cyclic, alkyl radicals which comprise 1 to 32 carbon atoms;
of 10 to 25% by weight, with respect to the total weight of the polycondensate, of at least one saturated or unsaturated, indeed even aromatic, linear, branched and/or cyclic, polycarboxylic acid comprising at least 2 COOH carboxyl groups, in particular 2 to 4 COOH groups; and/or a cyclic anhydride of such a polycarboxylic acid, and
b) at least one polyoxyalkylene block copolymer chosen from:
block copolymers of following formula (I):
HO—(CH₂—CH₂—O)ₙ—(CₓH₂ₓ—O)ₘ—(CH₂—CH₂—O)ₚ—H,
in which n, m and p are independently integers ranging from 1 to 1000 and x is an integer strictly greater than 2 and less than or equal to 10,
copolymers comprising at least one polyethylene glycol block and at least one polypropylene glycol block, the said copolymer exhibiting a molecular weight of greater than or equal to 1000 g/mol,
and their mixtures.

2. Composition according to claim 1, in which the polyol of the polycondensate comprises 3 to 4 hydroxyl groups.

3. Composition according to one of the preceding claims, in which the polyol of the polycondensate is a saturated or unsaturated, linear, branched and/or cyclic, carbon compound, in particular hydrocarbon compound, comprising 3 to 18 carbon atoms, in particular 3 to 12 carbon atoms, indeed even 4 to 10 carbon atoms, and 3 to 6 hydroxyl (OH) groups which can additionally comprise one or more oxygen atoms intercalated in the chain (ether functional group).

4. Composition according to one of the preceding claims, in which the polyol of the polycondensate is a saturated, linear or branched, hydrocarbon compound comprising 3 to 18 carbon atoms, in particular 3 to 12 carbon atoms, indeed even 4 to 10 carbon atoms, and 3 to 6 hydroxyl (OH) groups.

5. Composition according to one of the preceding claims, in which the polyol of the polycondensate is chosen, alone or as a mixture, from:
triols, such as 1,2,6-hexanetriol, trimethylolethane, trimethylolpropane or glycerol;
tetraols, such as pentaerythritol, erythritol, diglycerol or ditrimethylolpropane;
pentols, such as xylitol,
hexyls, such as sorbitol and mannitol; or also dipentaerythritol or triglycerol.

6. Composition according to one of the preceding claims, in which the polyol of the polycondensate is chosen from glycerol, pentaerythritol, sorbitol and their mixtures and better still is pentaerythritol.

7. Composition according to one of the preceding claims, in which the polyol or the polyol mixture of the polycondensate represents 16 to 28% by weight and better still 18 to 25% by weight, of the total weight of the polycondensate.

8. Composition according to one of the preceding claims, in which the nonaromatic monocarboxylic acid of the polycondensate is of formula RCOOH in which R is a saturated or unsaturated, linear, branched and/or cyclic, hydrocarbon radical comprising 5 to 31 carbon atoms, in particular 7 to 27 carbon atoms and better still 9 to 19 carbon atoms, indeed even 11 to 17 carbon atoms.

9. Composition according to claim 8, in which the R radical is saturated.

10. Composition according to either of claims 8 and 9, in which the R radical is linear or branched and preferably comprises 5 to 31 carbon atoms.

11. Composition according to one of the preceding claims, in which the nonaromatic monocarboxylic acid of the polycondensate is chosen, alone or as a mixture, from:
saturated monocarboxylic acids, such as caproic acid, caprylic acid, isoheptanoic acid, 4-ethylpentanoic acid, 2-ethylhexanoic acid, 4,5-dimethylhexanoic acid, 2-heptylheptanoic acid, 3,5,5-trimethylhexanoic acid, octanoic acid, isooctanoic acid, nonanoic acid, decanoic acid, isononanoic acid, lauric acid, tridecanoic acid, myristic acid, palmitic acid, stearic acid, isostearic acid, arachidic acid, behenic acid, cerotic (hexacosanoic) acid, cyclopentanecarboxylic acid, cyclopentaneacetic acid, 3-cyclopentylpropionic acid, cyclohexane-carboxylic acid, cyclohexylacetic acid or 4-cyclohexylbutyric acid;
unsaturated but nonaromatic monocarboxylic acids, such as caproleic acid, undecylenic acid, dodecylenic acid, myristoleic acid, palmitoleic acid, oleic acid, elaidic acid, gondoic acid or erucic acid.

12. Composition according to one of the preceding claims, in which the nonaromatic monocarboxylic acid of the polycondensate is chosen from 2-ethylhexanoic acid, isooctanoic acid, lauric acid, palmitic acid, isostearic acid and their mixtures and better still isostearic acid alone.

13. Composition according to one of the preceding claims, in which the nonaromatic monocarboxylic acid of the polycondensate or the mixture of the said acids represents 8 to 38% by weight and better still 10 to 35% by weight, of the total weight of the polycondensate.

14. Composition according to one of the preceding claims, in which the aromatic monocarboxylic acid of the polycondensate is of formula R′COOH in which R′ is an aromatic hydrocarbon radical comprising 6 to 10 carbon atoms and in particular the benzoic and naphthoic radicals, it being possible for the said R′ radical in addition to be substituted by 1 to 3 saturated or unsaturated, linear, branched and/or cyclic, alkyl radicals comprising 1 to 32 carbon atoms, in particular 2 to 12 carbon atoms, indeed even 3 to 8 carbon atoms, chosen in particular from methyl, ethyl, propyl, isopropyl, butyl, isobutyl, tert-butyl, pentyl, isopentyl, neopentyl, cyclopentyl, hexyl, cyclohexyl, heptyl, isoheptyl, octyl or isooctyl.

15. Composition according to one of the preceding claims, in which the aromatic monocarboxylic acid of the polycondensate is chosen, alone or as a mixture, from benzoic acid, o-toluic acid, m-toluic acid, p-toluic acid, 1-naphthoic acid, 2-naphthoic acid, 4-(tert-butyl)benzoic acid, 1-methyl-2-naphthoic acid or 2-isopropyl-1-naphthoic acid.

16. Composition according to one of the preceding claims, in which the aromatic monocarboxylic acid of the polycondensate is chosen from benzoic acid, o-toluic acid, m-toluic acid or 1-naphthoic acid, alone or as mixtures, and better still benzoic acid alone.

17. Composition according to one of the preceding claims, in which the aromatic monocarboxylic acid of the polycondensate or the mixture of the said acids represents 20 to 52% by weight, indeed even 22 to 52% by weight and better still 25 to 50% by weight, of the total weight of the polycondensate.

18. Composition according to one of the preceding claims, in which the polycarboxylic acid of the polycondensate is chosen from saturated or unsaturated, indeed even aromatic, linear, branched and/or cyclic, polycarboxylic acids comprising 2 to 20 carbon atoms, in particular 3 to 18 carbon atoms and better still 4 to 12 carbon atoms, indeed even 4 to 10 carbon atoms, the said acid comprising at least two COOH carboxyl groups, preferably from 2 to 4 COOH groups.

19. Composition according to claim 18, in which the said polycarboxylic acid of the polycondensate is saturated linear aliphatic and comprises 2 to 20 carbon atoms, in particular 3 to 18 carbon atoms, indeed even 4 to 12 carbon atoms.

20. Composition according to claim 18, in which the said polycarboxylic acid of the polycondensate is aromatic and comprises 8 to 12 carbon atoms.

21. Composition according to one of claims 1 to 17, in which the cyclic anhydride corresponds to one of the following formulae: 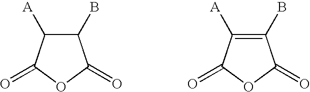
in which the A and B groups are, independently of one another:
a hydrogen atom,
a saturated or unsaturated, linear, branched and/or cyclic, aliphatic carbon radical or else an aromatic radical comprising 1 to 16 carbon atoms, in particular 2 to 10 carbon atoms, indeed even 4 to 8 carbon atoms, in particular methyl or ethyl;
or else A and B, taken together, form a saturated or unsaturated, indeed even aromatic, ring comprising, in total, 5 to 7 carbon atoms and in particular 6 carbon atoms.

22. Composition according to claim 21, in which A and B represent a hydrogen atom or together form an aromatic ring comprising a total of 6 carbon atoms.

23. Composition according to one of the preceding claims, in which the polycarboxylic acid of the polycondensate or its anhydride is chosen, alone or as a mixture, from:
dicarboxylic acids, such as decanedioic acid, dodecanedioic acid, cyclopropanedicarboxylic acid, cyclohexanedicarboxylic acid, cyclobutanedicarboxylic acid, naphthalene-1,4-dicarboxylic acid, naphthalene-2,3-dicarboxylic acid, naphthalene-2,6-dicarboxylic acid, suberic acid, oxalic acid, malonic acid, succinic acid, phthalic acid, terephthalic acid, isophthalic acid, pimelic acid, sebacic acid, azelaic acid, glutaric acid, adipic acid, fumaric acid or maleic acid;
tricarboxylic acids, such as cyclohexanetricarboxylic acid, trimellitic acid, 1,2,3-benzenetricarboxylic acid or 1,3,5-benzenetricarboxylic acid;
tetracarboxylic acids, such as butanetetracarboxylic acid and pyromellitic acid;
cyclic anhydrides of these acids and in particular phthalic anhydride, trimellitic anhydride, maleic anhydride and succinic anhydride.

24. Composition according to one of the preceding claims, in which the polycarboxylic acid of the polycondensate or its anhydride is chosen from phthalic anhydride and/or isophthalic acid and better still isophthalic acid alone.

25. Composition according to one of the preceding claims, in which the polycarboxylic acid of the polycondensate and/or its cyclic anhydride represents 11 to 22% by weight and better still 12 to 20% by weight, of the total weight of the polycondensate.

26. Composition according to one of the preceding claims, in which the polycondensate additionally comprises at least one silicone possessing a hydroxyl (OH) and/or carboxyl (COOH) functional group.

27. Composition according to claim 26, in which the silicone has a weight-average molecular weight (Mw) of between 300 and 20 000, in particular 400 and 10 000, indeed even 800 and 4000.

28. Composition according to either of claims 26 and 27, in which the silicone is of formula: 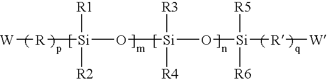
in which:
W and W′ are, independently of one another, OH or COOH; preferably, W═W′;
p and q are, independently of one another, equal to 0 or 1,
R are R′ are, independently of one another, a saturated or unsaturated, indeed even aromatic, linear, branched and/or cyclic, divalent carbon radical, in particular a hydrocarbon radical, comprising 1 to 12 carbon atoms, in particular 2 to 8 carbon atoms, and optionally comprising, in addition, one or more heteroatoms chosen from O, S and N, in particular O (ether);
in particular, R and/or R′ can be of formula —(CH₂)ₐ— with a=1-12, and in particular methylene, ethylene, propylene or phenylene;
or else of formula —[(CH₂)ₓO]— with x=1, 2 or 3 and z=1-10; in particular, x=2 or 3 and z=1-4; and better still x=3 and z=1.
R1 to R6 are, independently of one another, a saturated or unsaturated, indeed even aromatic, linear, branched and/or cyclic, carbon radical comprising 1 to 20 carbon atoms, in particular 2 to 12 carbon atoms; preferably, R1 to R6 are saturated or else aromatic and can in particular be chosen from alkyl radicals, in particular the methyl, ethyl, propyl, isopropyl, butyl, pentyl, hexyl, octyl, decyl, dodecyl and octadecyl radicals, cycloalkyl radicals, in particular the cyclohexyl radical, aryl radicals, in particular phenyl and naphthyl, arylalkyl radicals, in particular benzyl and phenylethyl, and also the tolyl and xylyl radicals.
m and n are, independently of one another, integers between 1 and 140 and are such that the weight-average molecular weight (Mw) of the silicone is between 300 and 20 000, in particular 400 and 10 000, indeed even between 800 and 4000.

29. Composition according to one of claims 26 to 28, in which the silicone is chosen, alone or as a mixture, from α,ω-diol or α,ω-dicarboxyl polyalkylsiloxanes and in particular α,ω-diol polydimethylsiloxanes and α,ω-dicarboxyl polydimethylsiloxanes; α,ω-diol or α,ω-dicarboxyl polyarylsiloxanes and in particular α,ω-diol or α,ω-dicarboxyl polyphenylsiloxanes; polyaryl-siloxanes possessing silanol functional groups, such as polyphenylsiloxane; polyalkylsiloxanes possessing silanol functional groups, such as polydimethylsiloxane; polyaryl/alkylsiloxanes possessing silanol functional groups, such as polyphenyl/methylsiloxane or polyphenyl/propylsiloxane.

30. Composition according to one of claims 26 to 29, in which the silicone is chosen from α,ω-diol polydimethylsiloxanes with a weight-average molecular weight (Mw) of between 400 and 10 000, indeed even between 500 and 5000 and in particular between 800 and 4000.

31. Composition according to one of claims 26 to 30, in which the silicone represents 0.1 to 15% by weight, in particular 1 to 10% by weight, indeed even 2 to 8% by weight, of the total weight of the polycondensate.

32. Composition according to one of the preceding claims, in which the ratio of the number of moles of aromatic monocarboxylic acid to the number of moles of nonaromatic monocarboxylic acid is between 1.2 and 8, in particular between 1.3 and 7.8, indeed even between 1.4 and 7.5 and better still between 1.9 and 7.

33. Composition according to one of the preceding claims, in which the polycondensate is capable of being obtained by reaction:
of at least one polyol chosen, alone or as a mixture, from 1,2,6-hexanetriol, trimethylolethane, trimethylolpropane, glycerol, pentaerythritol, erythritol, diglycerol, ditrimethylolpropane, xylitol, sorbitol, mannitol, dipentaerythritol and/or triglycerol; preferably present in an amount of 15 to 30% by weight, in particular 16 to 28% by weight and better still 18 to 25% by weight, with respect to the total weight of the final polycondensate;
of at least one nonaromatic monocarboxylic acid chosen, alone or as a mixture, from caproic acid, caprylic acid, isoheptanoic acid, 4-ethylpentanoic acid, 2-ethylhexanoic acid, 4,5-dimethylhexanoic acid, 2-heptylheptanoic acid, 3,5,5-trimethylhexanoic acid, octanoic acid, isooctanoic acid, nonanoic acid, decanoic acid, isononanoic acid, lauric acid, tridecanoic acid, myristic acid, palmitic acid, stearic acid, isostearic acid, arachidic acid, behenic acid, cerotic (hexacosanoic) acid, cyclopentane-carboxylic acid, cyclopentaneacetic acid, 3-cyclopentylpropionic acid, cyclohexane-carboxylic acid, cyclohexylacetic acid or 4-cyclohexylbutyric acid;
preferably present in an amount of 5 to 40% by weight, in particular 8 to 38% by weight and better still 10 to 35% by weight, with respect to the total weight of the final polycondensate;
of at least one aromatic monocarboxylic acid chosen, alone or as a mixture, from benzoic acid, o-toluic acid, m-toluic acid, p-toluic acid, 1-naphthoic acid, 2-naphthoic acid, 4-(tert-butyl)benzoic acid, 1-methyl-2-naphthoic acid or 2-isopropyl-1-naphthoic acid;
preferably present in an amount of 10 to 55% by weight, in particular 20 to 52% by weight and better still 25 to 50% by weight, with respect to the total weight of the final polycondensate; and
of at least one polycarboxylic acid or one of its anhydrides chosen, alone or as a mixture, from decanedioic acid, dodecanedioic acid, cyclopropanedicarboxylic acid, cyclohexanedicarboxylic acid, cyclobutanedicarboxylic acid, naphthalene-1,4-dicarboxylic acid, naphthalene-2,3-dicarboxylic acid, naphthalene-2,6-dicarboxylic acid, suberic acid, oxalic acid, malonic acid, succinic acid, phthalic acid, terephthalic acid, isophthalic acid, pimelic acid, sebacic acid, azelaic acid, glutaric acid, adipic acid, fumaric acid, maleic acid, cyclohexanetricarboxylic acid, trimellitic acid, 1,2,3-benzenetricarboxylic acid, 1,3,5-benzenetricarboxylic acid, butanetetracarboxylic acid, pyromellitic acid, phthalic anhydride, trimellitic anhydride, maleic anhydride and succinic anhydride;
preferably present in an amount of 10 to 25% by weight, in particular 11 to 22% by weight and better still 12 to 20% by weight, with respect to the total weight of the final polycondensate.

34. Composition according to one of the preceding claims, in which the polycondensate is capable of being obtained by reaction:
of at least one polyol chosen, alone or as a mixture, from glycerol, pentaerythritol, sorbitol and their mixtures and better still pentaerythritol alone, present in an amount of 15 to 30% by weight, in particular 16 to 28% by weight and better still 18 to 25% by weight, with respect to the total weight of the final polycondensate;
of at least one nonaromatic monocarboxylic acid chosen, alone or as a mixture, from 2-ethylhexanoic acid, isooctanoic acid, lauric acid, palmitic acid, isostearic acid and their mixtures and better still isostearic acid alone, present in an amount of 5 to 40% by weight, in particular 8 to 38% by weight and better still 10 to 35% by weight, with respect to the total weight of the final polycondensate;
of at least one aromatic monocarboxylic acid chosen, alone or as a mixture, from benzoic acid, o-toluic acid, m-toluic acid, 1-naphthoic acid and better still benzoic acid alone, present in an amount of 10 to 55% by weight, in particular 20 to 52% by weight and better still 25 to 50% by weight, with respect to the total weight of the final polycondensate; and
of at least one polycarboxylic acid or one of its anhydrides chosen, alone or as a mixture, from phthalic anhydride and isophthalic acid and better still isophthalic acid alone, present in an amount of 10 to 25% by weight, in particular 11 to 22% by weight and better still 12 to 20% by weight, with respect to the total weight of the final polycondensate.

35. Composition according to one of the preceding claims, in which the polycondensate exhibits at least one of the following characteristics:
an acid number, expressed as mg of potassium hydroxide per g of polycondensate, of greater than or equal to 8, in particular of between 8 and 40 and better still of between 10 and 30; and/or
a hydroxyl number, expressed as mg of potassium hydroxide per g of polycondensate, of greater than or equal to 30, in particular of between 30 and 100 and better still of between 40 and 90;
a viscosity, measured at 110° C., of between 75 and 6000 mPa·s, in particular between 80 and 5500 mPa·s, indeed even between 90 and 5000 mPa·s and better still between 200 and 4800 mPa·s;
a solubility in butyl acetate or ethyl acetate at 25° C. in a proportion of at least 50% by weight;
a viscosity of a solution of the polymer in butyl acetate or ethyl acetate at 25° C. at a concentration of 70% by weight of between 100 and 1500 mPa·s, in particular between 120 and 900 mPa·s.

36. Composition according to one of the preceding claims, in which the polycondensate is present in a content as dry matter ranging from 0.5 to 50% by weight, preferably from 0.5 to 40% by weight, in particular from 1 to 25% by weight and better still from 5 to 20% by weight, with respect to the weight of the final composition.

37. Composition according to claim 1, characterized in that the polyoxyalkylene block copolymer corresponds to the following formula:
HO—(CH₂—CH₂—O)ₙ—(CₓH₂ₓO)ₘ—H
with n and m integers between 1 and 1000 and x an integer strictly greater than 2 and less than or equal to 10.

38. Composition according to one of the preceding claims, characterized in that the polyoxyalkylene block copolymer is present in an amount ranging from 0.1 to 15% by weight, with respect to the total weight of the composition, preferably from 0.5 to 10% by weight and better still from 1 to 8% by weight.

39. Composition according to one of the preceding claims, characterized in that the organic solvent medium comprises at least one organic solvent chosen from:
ketones which are liquid at ambient temperature, such as methyl ethyl ketone, methyl isobutyl ketone, diisobutyl ketone, isophorone, cyclohexanone or acetone;
alcohols which are liquid at ambient temperature, such as ethanol, isopropanol, diacetone alcohol, 2-butoxyethanol or cyclohexanol;
propylene glycol ethers which are liquid at ambient temperature, such as propylene glycol monomethyl ether, propylene glycol monomethyl ether acetate or dipropylene glycol mono(n-butyl)ether;
cyclic ethers, such as γ-butyrolactone;
short-chain esters (having from 3 to 8 carbon atoms in total), such as ethyl acetate, methyl acetate, propyl acetate, isopropyl acetate, n-butyl acetate, isopentyl acetate, methoxypropyl acetate or butyl lactate;
ethers which are liquid at ambient temperature, such as diethyl ether, dimethyl ether or dichlorodiethyl ether;
alkanes which are liquid at ambient temperature, such as decane, heptane, dodecane or cyclohexane;
alkyl sulphoxides, such as dimethyl sulphoxide;
aldehydes which are liquid at ambient temperature, such as benzaldehyde or acetaldehyde;
ethyl 3-ethoxypropionate;
carbonates, such as propylene carbonate or dimethyl carbonate;
acetals, such as methylal;
and their mixtures.

40. Composition according to one of the preceding claims, characterized in that the organic solvent medium represents from 10 to 95% by weight, with respect to the total weight of the composition, preferably from 15% to 80% by weight and better still from 60 to 80% by weight.

41. Composition according to one of the preceding claims, characterized in that it comprises at least one thickening agent.

42. Composition according to claim 41, characterized in that the thickening agent is chosen from silicas, in particular hydrophobic silicas, clays, such as montmorillonite, modified clays, such as bentones, polysaccharide alkyl ethers and their mixtures.

43. Composition according to claim 41 or 42, characterized in that the thickening agent is present in a content ranging from 0.01 to 15% by weight, with respect to the total weight of the composition, preferably from 0.05 to 10% by weight and better still from 0.1 to 5% by weight.

44. Composition according to one of the preceding claims, characterized in that it comprises a film-forming polymer.

45. Composition according to the preceding claim, characterized in that the film-forming polymer is present in a content as dry matter ranging from 0.1% to 60% by weight, with respect to the total weight of the composition, preferably from 2% to 50% by weight and better still from 5% to 30% by weight.

46. Composition according to one of the preceding claims, characterized in that it comprises a colouring material.

47. Composition according to the preceding claim, characterized in that the colouring material is present in a content ranging from 0.01% to 50% by weight, with respect to the weight of the composition, preferably from 0.01% to 30% by weight.

48. Cosmetic method for making up or for the non-therapeutic care of the nails, comprising the application, to the nails, of at least one layer of a nail varnish composition according to one of claims 1 to 47.

49. Use of a nail varnish composition according to one of claims 1 to 47 for obtaining, after application to the nails, a glossy varnish film.

50. Cosmetic combination comprising: i) a container delimiting at least one compartment, the said container being closed by a closure element, and ii) a composition positioned inside the said compartment, the composition being in accordance with any one of claims 1 to 47.
